(19) United States
(12) Patent Application Publication
(10) Pub. No.: US 2017/0145452 A1
(43) Pub. Date: May 25, 2017

(54) CORYNEBACTERIUM GENUS MICROORGANISM FOR PRODUCING L-LYSINE AND METHOD FOR PRODUCING L-LYSINE BY USING THE SAME

(71) Applicant: CJ CHEILJEDANG CORPORATION, Seoul (KR)

(72) Inventors: Seung Bin LEE, Goyang-si, Gyeonggi-do (KR); Hyung Joon KIM, Seoul (KR); Lan HUR, Seoul (KR); Jun Ok MOON, Seoul (KR)

(21) Appl. No.: 15/309,921

(22) PCT Filed: Apr. 24, 2015
(86) PCT No.: PCT/KR2015/004092

§ 371 Date: Nov. 9, 2016

(30) Foreign Application Priority Data
May 14, 2014 (KR) 10-2014-0057966

(51) Int. Cl. C12P 13/08

(52) U.S. Cl. CPC C12P 13/08

(57) ABSTRACT

The present application relates to a microorganism of the genus Corynebacterium for producing L-lysine and a method for producing L-lysine by using the same. More specifically, the present application relates to: a recombinant microorganism of the genus Corynebacterium having increased L-lysine production, obtained by inactivating an intrinsic oxaloacetate decarboxylase of a Corynebacterium genus microorganism; and a method for producing L-lysine by using the same.

### TECHNICAL FIELD

The present application relates to a microorganism of the genus Corynebacterium producing L-lysine and a method for producing L-lysine using the same.

### BACKGROUND ART

L-lysine is biosynthesized from a precursor oxaloacetate via a lysine biosynthetic pathway, and oxaloacetate is degraded to pyruvate and carbon dioxide by oxaloacetate decarboxylase (odx).

Previously, Simon Klaffl and Bernhard J. Eikmanns simply demonstrated that a gene cg1458 encodes oxaloacetate decarboxylase by confirming that overexpression of the intrinsic gene cg1458 in Corynebacterium glutamicum increases oxaloacetate decarboxylase activity and also decreases L-lysine production, and the oxaloacetate decarboxylase activity is attenuated by inactivation of the gene, but they did not demonstrate changes in the L-lysine production by inactivation of cg1458 (Klaffl S, Eikmanns B J, et al., J. Bacteriol., May 2010, 192(10), p. 2604-12). However, the present inventors predicted that when activity of the intrinsic oxaloacetate decarboxylase is inhibited in an L-lysine-producing Corynebacterium strain, the supply of oxaloacetate for the L-lysine biosynthetic pathway is increased without consumption for oxaloacetate in other pathways, and they inactivated the oxaloacetate decarboxylase gene in different classes of L-lysine-producing strains. As a result, the present inventors found that inactivation of the oxaloacetate decarboxylase gene results in improving the L-lysine-producing ability of the strain, thereby completing the present application.

### DETAILED DESCRIPTION OF THE INVENTION

### Technical Problem

Accordingly, an object of the present application is to provide a microorganism of the genus Corynebacterium producing L-lysine.

Another object of the present application is to provide a method for producing L-lysine using the microorganism of the genus Corynebacterium.

### Technical Solution

In order to achieve the above objects, an aspect of the present application provides a microorganism of the genus Corynebacterium producing L-lysine, in which an oxaloacetate decarboxylase activity is inactivated.

Further, another aspect of the present application provides a method for producing L-lysine by culturing the microorganism of the genus Corynebacterium.

### Advantageous Effects of the Invention

The present application provides a recombinant microorganism of the genus Corynebacterium which has enhanced L-lysine production by inactivating an oxaloacetate decarboxylase gene in an L-lysine-producing Corynebacterium strain. The recombinant microorganism of the genus Corynebacterium is able to produce L-lysine with a high yield, thereby having industrially useful applications.

### DESCRIPTION OF THE DRAWINGS

FIG. 1 shows a pDZ-Δodx vector for deleting an odx gene in the chromosome of Corynebacterium.

### MODE OF THE INVENTION

Hereinafter, the present application will be described in detail.

An aspect of the present application provides a microorganism of the genus Corynebacterium which has enhanced L-lysine production by inactivating an oxaloacetate decarboxylase activity.

Oxaloacetate decarboxylase (odx) is an enzyme that catalyzes degradation of oxaloacetate to pyruvate and carbon dioxide, and an enzyme derived from any microorganism may be used, as long as the enzyme has the above activity. Specifically, an enzyme derived from a Coryneform microorganism, and more specifically, an enzyme derived from Corynebacterium glutamicum may be used, but is not limited thereto.

More specifically, the oxaloacetate decarboxylase of the present application may have an amino acid sequence of SEQ ID NO: 1, and may be encoded by the odx gene having a nucleotide sequence of SEQ ID NO: 2, but is not limited to the amino acid sequence or the nucleotide sequence. Specifically, a protein having 80% or higher, specifically 90% or higher, 95% or higher, and more specifically 97% or higher homology with the amino acid sequence of SEQ ID NO: 1 is also included in the scope of the present application, as long as the protein has the oxaloacetate decarboxylase activity. It is apparent that any amino acid sequence having a deletion, modification, substitution or addition of some sequence may be within the scope of the present application, as long as an amino acid sequence within the homology range has a biological activity that is substantially identical or corresponding to the protein of SEQ ID NO: 1. Any variant of the above nucleotide sequences encoding the same amino acid sequences, which results from genetic code degeneracy, may also be included in the present application.

The term ‘homology’, as used herein, refers to a degree of matching with a given amino acid sequence or nucleotide sequence, and the homology may be expressed as a percentage. In the present application, a homology sequence having an activity which is identical or similar to the given amino acid sequence or nucleotide sequence is expressed as “% homology”. The homology sequence may be determined by, for example, algorithm BLAST [see Karlin and Altschul, Pro. Natl. Acad. Sci. USA, 90, 5873(1993)] or FASTA by Pearson [see Methods Enzymol., 183, 63(1990)]. Based on this algorithm, a program called BLASTN or BLASTX has been developed [see http://www.ncbi.nlm.nih.gov]. The homology sequence may be also identified by comparing the sequences in a Southern hybridization experiment under stringent conditions as defined, and defining appropriate hybridization conditions are within the skill of the art (see e.g., Sambrook et al., 1989, infra.), and may be determined by a method well known to those skilled in the art.

The term ‘inactivation’, as used herein, means that the activity is further attenuated or eliminated, compared to a protein activity that occurs in a natural state or an unmodified state of an original microorganism, and the inactivation may be performed by any inactivation method known in the art. In the present application, inactivation of oxaloacetate decarboxylase means that the intrinsic odx gene is expressed at a low level or is not expressed, compared to that of a parent strain or an unmodified strain, or the activity is eliminated or decreased, even though the gene is expressed.

Specifically, the intrinsic activity of oxaloacetate decarboxylase may be inactivated by introducing a recombinant vector including a part of the gene encoding oxaloacetate decarboxylase into a microorganism to delete or mutate the intrinsic oxaloacetate decarboxylase gene, the expression of the protein may be attenuated by modifying a nucleotide sequence of a promoter region or 5′-UTR region of the gene, or the activity of the protein may be attenuated by introducing a mutation into an ORF region of the corresponding gene. Further, insertion of the gene into the chromosome may be performed by any method known in the art.

The term ‘intrinsic activity’, as used herein, refers to a protein activity of an original microorganism in a natural state or an unmodified state.

More specifically, the inactivation may be caused by site-specific gene disruption, but is not limited thereto.

The term ‘recombinant vector’, as used herein, refers to a DNA construct including a polynucleotide sequence of a polynucleotide encoding a target protein, which is operably linked to a suitable regulatory sequence so that the target protein may be expressed in an appropriate host. The regulatory sequence may include a promoter capable of initiating transcription, any operator sequence for regulating the transcription, a sequence encoding a suitable mRNA ribosome-binding site, and a sequence regulating termination of transcription and translation. The vector, after being transformed into a suitable host cell, may be replicated or function irrespective of the host genome, or may be integrated into the genome itself. The vector used in the present application is not particularly limited, as long as the vector is replicable in the host, and any vector known in the art may be used.

The term ‘transformation’, as used herein, means that a gene is introduced into a host cell, thereby enabling the expression of the gene in the host cell. The transformed gene may be located inside or outside the chromosome of the host cell, as long as it may be expressed in the host cell. The gene may be introduced in any form, as long as it may be introduced into the host cell and expressed therein.

As a parent cell into which the recombinant vector is introduced, any microorganism may be used without limitation, as long as the microorganism is able to produce L-lysine. Specifically, a microorganism of the genus Corynebacterium or the genus Brevibacterium, and more specifically, Corynebacterium glutamicum may be used, but is not limited thereto.

In a specific embodiment of the present application, a recombinant vector was constructed by inserting a part of the odx gene into a pDZ vector (Korean Patent No. 0924065) which is not replicable in Corynebacterium glutamicum, and transformation of the recombinant vector into a microorganism and homologous recombination of the recombinant vector into the chromosome were allowed to prepare the microorganism of the genus Corynebacterium with enhanced L-lysine production, in which oxaloacetate decarboxylase is inactivated, but the present application is not limited thereto.

All the Corynebacterium glutamicum strains prepared in an embodiment of the present application showed increased L-lysine production, compared to the parent strain, indicating that they are able to produce L-lysine with a high yield by inactivation of the odx gene.

Another aspect of the present application provides a method for producing L-lysine by culturing the microorganism of the genus Corynebacterium. In more detail, provided is a method for producing L-lysine, the method including producing L-lysine in a culture or cells of the microorganism by culturing the microorganism of the present application; and recovering L-lysine from the culture or cells of the microorganism.

In the method of the present application, the culturing of the microorganism of the genus Corynebacterium may be performed by using any culture conditions and methods known in the art, but is not limited to the following example.

A medium which may be used for the culturing of the microorganism of the genus Corynebacterium may be exemplified by media disclosed in Manual of Methods for General Bacteriology by the American Society for Bacteriology (Washington D.C., USA, 1981).

Carbon sources to be used in the medium may include saccharides and carbohydrates such as glucose, sucrose, lactose, fructose, maltose, starch and cellulose, oils and lipids such as soybean oil, sunflower seed oil, castor oil and coconut oil, fatty acids such as palmitic acid, stearic acid, and linoleic acid, alcohols such as glycerol and ethanol, and organic acids such as acetic acid. These materials may be used separately or in a mixture.

Nitrogen sources to be used may include peptone, yeast extract, meat broth, malt extract, corn steep liquor, soybean meal and urea, or inorganic compounds, for example, ammonium sulfate, ammonium chloride, ammonium phosphate, ammonium carbonate and ammonium nitrate. These nitrogen sources may be also used separately or in a mixture.

Phosphorus sources to be used may include dipotassium hydrogen phosphate, potassium dihydrogen phosphate, or corresponding sodium-containing salts, and the culture medium may include metal salts such as magnesium sulfate or iron sulfate required for the growth. In addition to these materials, essential growth materials such as amino acids and vitamins may be used. Further, proper precursors may be added to the culture medium. These materials may be added to the culture during cultivation by an appropriate method in a batch or continuous mode.

During the culturing of the microorganism, pH of the culture may be adjusted with a basic compound such as sodium hydroxide, potassium hydroxide or ammonia, or an acidic compound such as phosphoric acid or sulfuric acid. The generation of foam may be restrained using an anti-foaming agent such as fatty acid polyglycol ester. To maintain aerobic conditions, oxygen or an oxygen-containing gas (e.g., air) may be introduced into the culture. The culture temperature may be generally between 20° C. and 45° C., and specifically, between 25° C. and 40° C. The culturing may be continued until a desired amount of L-lysine is produced. Specifically, the culturing time may be 10 hrs to 160 hrs.

In the method of the present application, the culturing may be performed in a continuous or batch mode such as a batch process, a fed-batch process, and a repeated fed-batch process. The culturing method is well known in the art, and any method may be used.

Hereinafter, the present application will be described in more detail with reference to Examples. However, these Examples are for illustrative purposes only, and the scope of the present application is not intended to be limited by these Examples.

### EXAMPLE

### Example 1

### Construction of Recombinant Vector for Inactivation of Corynebacterium-Derived odx Gene

In order to inactivate an odx gene (SEQ ID NO: 2) encoding oxaloacetate decarboxylase of Corynebacterium glutamicum having an amino acid sequence represented by SEQ ID NO: 1 by site-specific gene disruption, a plasmid vector was constructed by the following method. First, based on NIH Genbank (USA), a nucleotide sequence of odx gene was obtained, and on the basis of this sequence, primers for the preparation of inactivated fragments of the odx gene were synthesized.

PCR[Sambrook et al, Molecular Cloning, a Laboratory Manual (1989), Cold Spring Harbor Laboratories] was performed using the chromosome of the wild-type Corynebacterium glutamicum ATCC13032 as a template and primers of SEQ ID NO: 3 and SEQ ID NO: 4, SEQ ID NO: 5 and SEQ ID NO: 6. After denaturation at 95° C. for 5 minutes, 30 cycles of denaturation at 95° C. for 30 seconds, annealing at 55° C. for 30 seconds, and polymerization at 72° C. for 30 seconds were repeated, and then polymerization at 72° C. for 7 minutes was performed. As a result, SEQ ID NO: 7 of 536 bp and SEQ ID NO: 8 of 534 bp including the 5′-end and the 3′-end of the odx gene, respectively were obtained.

SEQ ID NO: 3:
5′-TCTAGAACGATGAAATCGCCGCGGGT-3′
SEQ ID NO: 4:
5′-GGGTTGCCCAGCTTGCCGATCACGGGCGGTGAGGTTAGCT-3′
SEQ ID NO: 5:
5′-AGCTAACCTCACCGCCCGTGATCGGCAAGCTGGGCAACCC-3′
SEQ ID NO: 6:
5′-TCTAGAGGTTGCTGCGGATTCTGATT-3′

PCR was performed using the amplified SEQ ID NO: 7 and SEQ ID NO: 8 as a template and SEQ ID NO: 3 and SEQ ID NO: 6. After denaturation at 95° C. for 5 minutes, 30 cycles of denaturation at 95° C. for 30 seconds, annealing at 55° C. for 30 seconds, and polymerization at 72° C. for 60 seconds were repeated, and then polymerization at 72° C. for 7 minutes was performed. As a result, SEQ ID NO: 9 (hereinbelow, referred to as Δodx) of 1030 bp, in which the 5′-end and the 3′-end of the odx gene were linked to each other, was amplified.

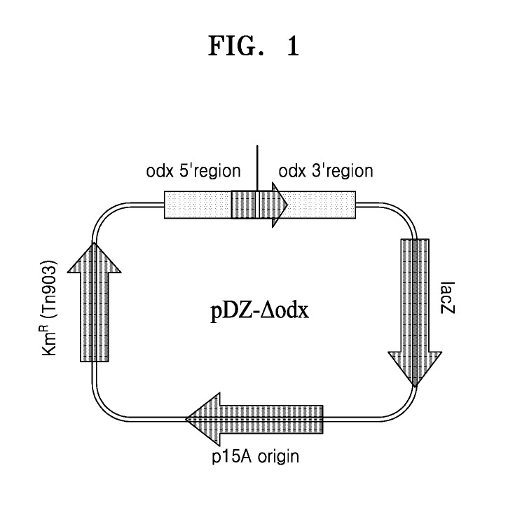

A pDZ vector (Korean Patent No. 0924065) which is not replicable in Corynebacterium glutamicum and the amplified DNA fragment Δodx were treated with a restriction enzyme XbaI, respectively and then, ligated with each other using DNA ligase. Cloning was performed to obtain a plasmid. This plasmid was designated as pDZ-Δodx (FIG. 1).

### Example 2

### Inactivation of odx in L-Lysine-Producing Corynebacterium and Analysis of L-Lysine Production

pDZ-Δodx prepared in Example 1 was transformed into L-lysine-producing strains, Corynebacterium glutamicum KCCM11016P and KCCM11347P by an electric pulse method (Appl. Microbiol. Biotechnol.(1999) 52:541-545), respectively and transformed strains were obtained on a selection medium containing 25 mg/L kanamycin. Strains having inactivated odx gene were obtained by the DNA fragment Δodx inserted into the genome by secondary recombination (cross-over), and the strains were designated as KCCM11016P/Δodx and KCCM11347P/Δodx, respectively.

In order to examine whether inactivation of the odx gene actually influences an increase in lysine production, the odx-inactivated strains, KCCM11016P/Δodx and KCCM11347P/Δodx were cultured by the following method and their production was examined.

Each of the strains was inoculated in a 250 ml corner-baffled flask containing 25 ml of the following seed medium, followed by culturing at 37° C. for 20 hours under stirring at 200 rpm. 1 mL of the seed culture was inoculated in a 250 ml corner-baffled flask containing 24 ml of the following production medium, followed by culturing at 30° C. for 72 hours under stirring at 200 rpm.

Composition of Seed Medium (pH 7.0):

20 g of glucose, 10 g of peptone, 10 g of yeast extract, 5 g of urea, 4 g of KH₂PO₄, 8 g of K₂HPO₄, 0.5 g of MgSO₄.7H₂O, 100 μg of biotin, 1000 μg of thiamine HCl (in 1 liter of process water)

Composition of Production Medium (pH 7.0):

80 g of glucose, 20 g of molasse or pretreated molasse (as reducing sugar), 5 g of corn steep liquor, 40 g of (NH₄)₂SO₄, 4 g of urea, 1 g of KH₂PO₄, 2.5 g of NaCl, 1 g of MgSO₄.7H₂O, 10 mg of FeSO₄.7H₂O, 10 mg of MnSO₄.5H₂O, 100 μg of biotin, 200 μg of thiamine HCl, 40 g of CaCO₃, if necessary, 0.4 g of L-leucine, if necessary, 0.1 g of L-threonine, if necessary, 0.1 g of L-methionine (in 1 liter of process water)

After completion of the culturing, L-lysine production was measured by HPLC. As a result, the content of L-lysine in the culture of each strain was as in the following Table 1.

TABLE 1

| | | L-lysine (g/l) | | | |
|---|---|---|---|---|---|
| | Strain | Batch 1 | Batch 2 | Batch 3 | Mean |
| Control | KCCM11016P | 43.1 | 43.0 | 43.0 | 43.0 |
| group | | | | | |
| Experimental | KCCM11016P/Δodx | 44.8 | 44.8 | 44.9 | 44.8 |
| group | | | | | |
| Control | KCCM11347P | 38.1 | 38.1 | 37.9 | 38.0 |
| group | | | | | |
| Experimental | KCCM11347P/Δodx | 39.5 | 39.6 | 39.6 | 39.6 |
| group | | | | | |

As shown in Table 1, odx-inactivated strains, KCCM11016P/Δodx and KCCM11347P/Δodx showed about 4.2% increase in L-lysine production, compared to their parent strains, KCCM11016P and KCCM11347P, respectively.

Of them, the strain KCCM11016P/Δodx was designated as CA01-2276, and deposited in the international depository authority, Korean Culture Center of Microorganisms, which is the Subsidiary Culture Collection of the Korean Federation of Culture Collections located at 361-221, Hongje-l-Dong, Seodaemungu-Gu, Seoul, Korea, on Nov. 22, 2013 under Accession No. KCCM11478P.

### Example 3

### Inactivation of odx in L-Lysine Biosynthetic Pathway-Enhanced, L-lysine-Producing Corynebacterium and Analysis of Lysine Production

pDZ-Δodx prepared in Example 1 was transformed into an L-lysine biosynthetic pathway-enhanced, L-lysine-producing strain, Corynebacterium glutamicum KCCM10770P (Korean Patent No. 10-0924065) in the same manner as in Example 2 to prepare a transformed strain, which was designated as KCCM10770P/Δodx.

In order to examine whether inactivation of the odx gene actually influences an increase in L-lysine production, the odx-inactivated strain, KCCM10770P/Δodx was cultured in the same manner as in Example 2, and its production was examined. The content of L-lysine in the culture of each strain was as in the following Table 2.

TABLE 2

| | | L-lysine (g/l) | | | |
|---|---|---|---|---|---|
| | Strain | Batch 1 | Batch 2 | Batch 3 | Mean |
| Control | KCCM10770P | 47.0 | 47.1 | 46.9 | 47.0 |
| group | | | | | |
| Experimental | KCCM10770P/Δodx | 50.1 | 50.0 | 50.0 | 50.0 |
| group | | | | | |

As shown in Table 2, the odx-inactivated strain, KCCM10770P/Δodx showed about 6.4% increase in L-lysine production, compared to the parent strain, KCCM10770P.

### Example 4

### Inactivation of odx in Wild-Type-Derived L-Lysine-Producing Corynebacterium and Analysis of Lysine Production

pDZ-Δodx prepared in Example 1 was transformed into a wild-type-derived L-lysine-producing strain, Corynebacterium glutamicum CJ3P (Binder et al. Genome Biology 2012, 13:R40, pyc(P458S), hom(V59A), lysC(T311I)) in the same manner as in Example 2 to prepare a transformed strain, which was designated as CJ3P/Δodx.

In order to examine whether inactivation of the odx gene actually influences an increase in lysine production, the odx-inactivated strain, CJ3P/Δodx was cultured in the same manner as in Example 2, and its production was examined. The content of L-lysine in the culture of each strain was as in the following Table 3.

TABLE 3

| | | L-lysine (g/l) | | | |
|---|---|---|---|---|---|
| | Strain | Batch 1 | Batch 2 | Batch 3 | Mean |
| Control group | CJ3P | 7.9 | 8.0 | 8.0 | 8.0 |
| Experimental | CJ3P/Δodx | 8.2 | 8.3 | 8.3 | 8.3 |
| group | | | | | |

As shown in Table 3, when odx was inactivated in the parent strain CJ3P which is the wild-type-derived L-lysine producing strain, about 3.8% increase in the lysine production was also observed.

Accordingly, the present applicants demonstrated in Examples 2 to 4 that increased lysine production was commonly observed in different classes of microorganisms of the genus Corynebacterium by inactivation of the odx gene, indicating that inactivation of the odx gene is an effective trait in the production of L-lysine.

1. A microorganism of the genus Corynebacterium producing L-lysine, wherein oxaloacetate decarboxylase having an amino acid sequence set forth in SEQ ID NO: 1 is inactivated.

2. The microorganism of the genus Corynebacterium according to claim 1, wherein the microorganism of the genus Corynebacterium is Corynebacterium glutamicum.

3. A method for producing L-lysine, the method comprising:
culturing the microorganism of claim 1 to obtain a culture or cells of the microorganism; and
recovering L-lysine from the culture or cells of the microorganism.

4. A method for producing L-lysine, the method comprising:
culturing the microorganism of claim 2 to obtain a culture or cells of the microorganism; and
recovering L-lysine from the culture or cells of the microorganism.


SEQUENCE LISTING (9 sequences)

SEQ ID NO: 1 - protein, 268 aa, Corynebacterium glutamicum
MRFGRIATPDGMCFCSIEGEGDDVANLTAREIEGTPFTEPKFTGREWPLKDVRLLAPMLPSKVVAIGRNYADHVAEVFKKSAESLPPTLFLKPPTAVTGPESPIRIPSFATKVEFEGELAVVIGKPCKNVKADDWKSVVLGFTIINDVSSRDLQFADGQWARAKGIDTFGPIGPWIETDINSIDLDNLPIKARLTHDGETQLKQDSNSNQMIMKMGEIIEFITASMTLLPGDVIATGSPAGTEAMVDGDYIEIEIPGIGKLGNPVVDA

SEQ ID NO: 2 - DNA, 807 bp, Corynebacterium glutamicum
atgcgttttggacgaattgccaccccagatggaatgtgtttttgctcaattgaaggcgaaggcgatgatgtagctaacctcaccgcccgtgagatcgagggtacccccttcactgaaccaaagttcaccggtcgcgaatggccacttaaagatgtacgcctgctggcaccaatgctgccaagcaaagtcgtcgcgattggccgtaactacgccgatcacgttgctgaggtgtttaagaagtccgcagaatcactgccaccaaccctgttcctgaagccaccaacagctgtcaccggaccagagtccccaatccgaatcccttcctttgccaccaaggtggaattcgaaggtgagctcgcagtagttatcggcaagccctgcaagaacgtcaaggctgatgactggaagtctgtcgttttgggcttcaccatcatcaacgacgtctcctcccgtgacctccagttcgctgacggccagtgggcacgcgctaagggcattgacaccttcggccccatcggaccatggattgaaactgacatcaactccatcgacttggacaacctgcccatcaaggcacgcctcacccacgacggcgaaacccaattgaagcaggactccaactccaaccagatgatcatgaagatgggcgaaattatcgagttcatcaccgcctccatgaccctgctcccaggcgacgttattgcaaccggttctccagcaggcaccgaagcaatggttgacggcgactacatcgaaatcgaaattccaggcatcggcaagctgggcaacccagttgtggacgcctaa

SEQ ID NO: 3 - DNA, 26 bp, Artificial Sequence
  primer
tctagaacgatgaaatcgccgcgggt

SEQ ID NO: 4 - DNA, 40 bp, Artificial Sequence
  primer
gggttgcccagcttgccgatcacgggcggtgaggttagct

SEQ ID NO: 5 - DNA, 40 bp, Artificial Sequence
  primer
agctaacctcaccgcccgtgatcggcaagctgggcaaccc

SEQ ID NO: 6 - DNA, 26 bp, Artificial Sequence
  primer
tctagaggttgctgcggattctgatt

SEQ ID NO: 7 - DNA, 536 bp, Artificial Sequence
  odx 5′ terminal
tctagaacgatgaaatcgccgcgggtatcgacatcaaagaggcgcttggccaactactgcaggcgctccagggacgcgccatgctggcgcatttttcacccatcgagcgcgattttatttcggccgcatgcctcaagcatttcggaacgcttctcgacgtccccctcgtggatacttttgccatggaacgccgccacatggagaggatgtccacctacccacgtggcgaagacctccggcttgcacgcattcgacaacgatatggtcttcccaattactccaatcaccaggcattaaccgacgctttggcgtgcgccgaggtgtatttggtgcagatcacgcaccttcgggcgaacaccctcaaagatatttgggaataggggtcatcgtgcaggcccccacacccaacgcgtaacataacggtcatgcgttttggacgaattgccaccccagatggaatgtgtttttgctcaattgaaggcgaaggcgatgatgtagctaacctcaccgcccgtgatcggcaagctgggcaaccc

SEQ ID NO: 8 - DNA, 534 bp, Artificial Sequence
  odx 3′ terminal
agctaacctcaccgcccgtgatcggcaagctgggcaacccagttgtggacgcctaaaatggatcaccaccatcacgatcatggggagaagcgtccgtacgaccaggtcaccccagaatccatggagaatcaatacaccaccacggaacaagtctggagcggcaaccccaaccaagcactcatcgcctacttgggtgaagagccagctgaaggcaccgcactagacattggctgtggcgaaggcgctgacctcgtgtggctgtcctccaagggatacgtcaccaccggcatcgacttcgcaccaactgcagtgaagcgcgcgaaacaaaacgccccatcagccgaagtcctcttggtatcattcaccgaattcgccatcgaatccggtcgccaattcgacctcgtcagctgctcgtacggccaaatcccagccaacgacacctccgtcgcgcaactggaaaacctcgtcgcacccggcggaaccctcctgttcacccaccacgactttgaatcagaatccgcagcaacctctaga

SEQ ID NO: 9 - DNA, 1030 bp, Artificial Sequence
  odx
tctagaacgatgaaatcgccgcgggtatcgacatcaaagaggcgcttggccaactactgcaggcgctccagggacgcgccatgctggcgcatttttcacccatcgagcgcgattttatttcggccgcatgcctcaagcatttcggaacgcttctcgacgtccccctcgtggatacttttgccatggaacgccgccacatggagaggatgtccacctacccacgtggcgaagacctccggcttgcacgcattcgacaacgatatggtcttcccaattactccaatcaccaggcattaaccgacgctttggcgtgcgccgaggtgtatttggtgcagatcacgcaccttcgggcgaacaccctcaaagatatttgggaataggggtcatcgtgcaggcccccacacccaacgcgtaacataacggtcatgcgttttggacgaattgccaccccagatggaatgtgtttttgctcaattgaaggcgaaggcgatgatgtagctaacctcaccgcccgtgatcggcaagctgggcaacccagttgtggacgcctaaaatggatcaccaccatcacgatcatggggagaagcgtccgtacgaccaggtcaccccagaatccatggagaatcaatacaccaccacggaacaagtctggagcggcaaccccaaccaagcactcatcgcctacttgggtgaagagccagctgaaggcaccgcactagacattggctgtggcgaaggcgctgacctcgtgtggctgtcctccaagggatacgtcaccaccggcatcgacttcgcaccaactgcagtgaagcgcgcgaaacaaaacgccccatcagccgaagtcctcttggtatcattcaccgaattcgccatcgaatccggtcgccaattcgacctcgtcagctgctcgtacggccaaatcccagccaacgacacctccgtcgcgcaactggaaaacctcgtcgcacccggcggaaccctcctgttcacccaccacgactttgaatcagaatccgcagcaacctctaga